(19) United States
(12) Patent Application Publication
(10) Pub. No.: US 2026/0083648 A1
(43) Pub. Date: Mar. 26, 2026

(54) DEODORANT COMPOSITIONS AND METHODS OF MAKING SAME

(71) Applicant: DR. SQUATCH, LLC, MARINA DEL REY, CA (US)

(72) Inventors: Alex URUCHURTU, Los Angeles, CA (US); Duane KLEINGARN, Marina Del Rey, CA (US); Alexandra BLACKSTONE, Marina Del Rey, CA (US)

(73) Assignee: DR. SQUATCH, LLC, MARINA DEL REY, CA (US)

(21) Appl. No.: 19/203,670

(22) Filed: May 9, 2025

Related U.S. Application Data
(60) Provisional application No. 63/648,180, filed on May 16, 2024.
(60) Provisional application No. 63/648,181, filed on May 16, 2024.
(60) Provisional application No. 63/734,219, filed on Dec. 16, 2024.
(60) Provisional application No. 63/753,707, filed on Feb. 4, 2025.

(51) Int. Cl. A61K 8/19; A61K 8/02; A61K 8/37; A61K 8/73; A61K 8/92; A61Q 15/00

(52) U.S. Cl. CPC A61K 8/19; A61K 8/0229; A61K 8/37; A61K 8/732; A61K 8/922; A61K 8/927; A61Q 15/00; A61K 2800/31; A61K 2800/34

(57) ABSTRACT

Deodorant compositions are described herein, in particular that form a stick deodorant. The deodorant compositions includes about 1% wt/wt beeswax to about 12% wt/wt beeswax; about 10% wt/wt or less coconut oil; about 1% wt/wt to about 10% wt/wt of an esterified oil; less than about 16% caprylic/capric triglyceride; at least 20% wt/wt of a starch; a deodorant agent; and a second wax selected from the group consisting of a glyceryl stearate, lauryl laurate, behenyl alcohol, and combinations thereof. The deodorant composition as a whole has at least 95% by weight natural or naturally-derived ingredients.

### TECHNICAL FIELD

This application relates to deodorant compositions, and more particularly to deodorant compositions in the form of sticks or pastes that are topically applicated to skin and that contain at least 95% by weight of natural or natural-derived ingredients, including but not limited to beeswax, coco-caprylate, shea butter, and coconut oil.

### BACKGROUND

Stick deodorants are a standard form of delivery of deodorant that is generally popular with consumers. Deodorants are a category of cosmetic products intended to mask and/or counteract unwanted human body odor. Deodorants typically contain antimicrobials and/or fragrances to inhibit the formation and/or perception of body odor. Antiperspirants are a subcategory of deodorants. Like deodorants, they are known to contain fragrances and/or antimicrobials, but an additional active ingredient(s), historically an aluminum or zinc salt, is included that blocks sweat pores. The presence of aluminum in antiperspirants has come under scrutiny in the past twenty years as having a correlation to increased risk of Alzheimer's disease, breast cancer, and/or prostate cancer.

In response, consumers have begun to desire more natural ingredients in products, and deodorants are no exception. Beyond aluminum, there are other chemicals to be avoided in deodorants as part of the natural ingredient trend: triclosan, phthalates, parabens, some non-natural fragrances, silicones, triethylamine, diethanolamine, FD&C colors, and talcum powder.

Stick deodorants need to have desirable properties, such as body integrity (not crumbling during application), glide without sticking to hair (it is undesirable for the deodorant to pull the user's underarm hair), a suitable melting point, a smooth solid body (i.e., no cracks), and shelf stability. Getting these properties balanced is needed to form a product that is acceptable to consumers, especially when natural ingredients are used, rather than substances such as silicones or siloxanes (e.g., dimethicone, cyclomethicones, etc.). In general, when there is a high powder content in a stick deodorant, it is a challenge to achieve desirable glide.

Besides stick deodorants and paste deodorants are becoming more generally acceptable to consumers and the above issues with appearance, glide, and the like can be avoided.

There is always a need for improved deodorant compositions that have natural ingredients in various forms, such as a stick deodorant and a paste deodorant.

### SUMMARY

In one aspect, deodorant compositions are disclosed that, as a whole, have at least 95% by weight natural or naturally-derived ingredients. The composition can be in the form of a stick deodorant or a paste deodorant and include about 1% wt/wt beeswax to about 12% wt/wt beeswax; about 10% wt/wt or less coconut oil; about 1% wt/wt to about 10% wt/wt of an esterified oil; less than about 18% wt/wt caprylic/capric triglyceride; at least 20% wt/wt of a starch, a deodorant agent, and a second wax selected from the group consisting of a glyceryl stearate, lauryl laurate, behenyl alcohol, and combinations thereof. The esterified oil can be coco-caprylate and/or isopropyl palmitate. When the esterified oil is a mixture of coco-caprylate and isopropyl palmitate, they are present in a range of ratios, respectively, of 1:1 to 1:2, or more preferably 1:1.1 to 1:1.4. In all embodiments, the deodorant is free of or substantially free of silicones and water. The caprylic/capric triglyceride can be about 10% wt/wt to about 16% wt/wt of the deodorant composition.

In all embodiments, the second wax can be a combination of glyceryl stearate, lauryl laurate, and behenyl alcohol, and the glyceryl stearate, lauryl laurate, and behenyl alcohol can be present in a ratio range of 3:1:1 to about 3:1.5:1. In one embodiment, the second wax is glyceryl stearate and/or lauryl laurate, and can additionally include jojoba ester. In all embodiments, the deodorant compositions can include an ester wax and greater than 2% wt/wt of a fatty alcohol. In some embodiments, the ester wax is glyceryl stearate, in other embodiments, the ester wax is lauryl laurate, and in other embodiments the ester wax comprises both glyceryl stearate and lauryl laurate. In all embodiments, the fatty alcohol is selected from the group consisting of behenyl alcohol, cetyl alcohol, oleyl alcohol, arachidyl alcohol, cetyl alcohol, and combination thereof.

In some embodiments, the concentration of caprylic/capric triglyceride is at least about 10% wt/wt of the deodorant composition, and the concentration of the coconut oil is about 4% wt/wt to 10% wt/wt of the deodorant composition. In all embodiments, the starch can be tapioca starch and/or arrowroot powder.

In all embodiments, the deodorant composition can include one or more of jojoba seed oil, jojoba esters, shea butter, triethyl citrate, tocopherol, maltodextrin Lactobacillus ferment, a lubricating powder, and a fragrance. The shea butter can be present in a range of about 1% wt/wt to about 8% wt/wt of the deodorant composition. The fragrance is present it can be 0.1% wt/wt to about 6% wt/wt of the deodorant composition.

In all embodiments, the deodorant agent can be selected from the group consisting of beryllium carbonate, magnesium carbonate, calcium carbonate, magnesium hydroxide, magnesium hydroxide and magnesium carbonate hydroxide, magnesium stearate, partially carbonated magnesium hydroxide, piroctone olamine, potassium carbonate, potassium bicarbonate, sodium carbonate, sodium sesquicarbonate, sodium bicarbonate, hexamidine, zinc carbonate, thymol, polyvinyl formate, salicylic acid, niacinamide, magnesium carbonate hydroxide, cinnamon essential oil, cinnamon bark essential oil, cinnamic aldehyde, magnesium oxide, zinc oxide, zinc stearate, and mixtures thereof.

### BRIEF DESCRIPTION OF THE DRAWINGS

FIG. 1 is a flow chart of a first method of making compositions disclosed herein.

FIG. 2 is a flow chart of a second method of making compositions disclosed herein.

FIG. 3 is a chart of example deodorant compositions.

FIG. 4 is a photograph showing a comparison of a deodorant stick that is cracked and that stuck to the container during advancement versus a commercially acceptable deodorant stick.

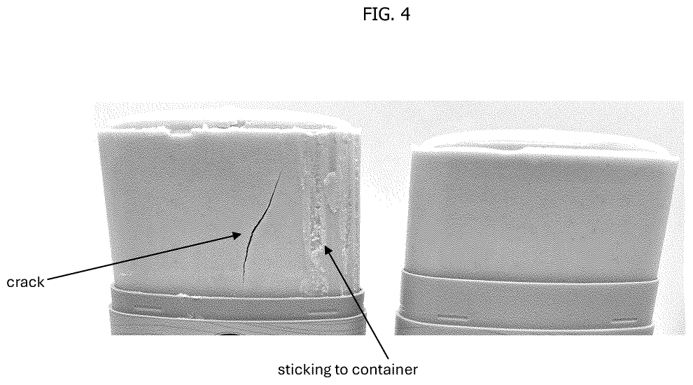

### DETAILED DESCRIPTION

In all aspects, deodorant compositions are disclosed herein. Preferably, the deodorant compositions form a stick deodorant or a paste deodorant. Moreover, the compositions utilize natural and/or naturally derived ingredients and are optionally fragranced. As used herein, a natural ingredient is an ingredient obtained only from a natural source such as a plant, animal, micro-organism, or mineral; however, ingredients obtained from fossil fuels are outside the definition of a natural ingredient. Natural ingredients include ingredients obtained from natural sources through physical processes, fermentation reactions occurring in nature and leading to molecules which occur in nature, and other procedures of preparation that do not involve intentional chemical modification. (International Organization for Standardization (ISO) 16128-1). A naturally derived ingredient is an ingredient of greater than 50% natural origin, by molecular weight, by renewal carbon content, by other methods used in the art, or obtained through chemical and/or biological processes resulting in chemical modification. (ISO 16128-1). Chemical modifications falling within the scope of naturally derived ingredients include, for example, saponification, neutralization, hydration, etherification, amination, esterification, amidation, and hydrolysis. The relevant chemical structure of the natural ingredient “remaining intact” means, by way of example, that the fatty chain acyl structure of a fatty acid remains after esterification or amidation. The above is consistent with the definition of “natural” as calculated by the ISO 16128-1 and/or ISO 16128-2 methodologies/mathematical formulations set forth therein. (ISO 16128-1 (2016) Guidelines on technical definitions and criteria for natural and organic cosmetic ingredients and products—Part 1: Definitions for ingredients, and ISO 16128-2 (2017) Guidelines on technical definitions and criteria for natural and organic cosmetic ingredients and products—Part 2: Criteria for ingredients and products, both of which are hereby incorporated herein by reference in their entirety.)

“Natural” or “naturally-derived” is applied herein to substances, ingredients, and products, and may collectively be referred to as natural substances, natural ingredients, and natural products. In other words, the use of the phrase “natural” herein is understood to mean natural and/or naturally derived in all instances. The natural content of a formulation can be calculated based on the ISO methodologies noted above, especially ISO 16128-2 (natural origin). Each ingredient, taken separately, meets the definition of a naturally derived ingredient if it is an ingredient having greater than 50% natural origin. The natural content of a formulation is the mass percentage of all natural or naturally-derived ingredients present therein. It is calculated as the sum of the relative concentrations of each ingredient in the formulation multiplied by each ingredient's respective percentage of natural origin. Again, this is explained in detail in ISO 16218-2(5.2). Each deodorant composition disclosed herein as a whole is composed of at least 95% natural ingredients, more preferably at least 98% natural ingredients.

All numerical values set forth herein can be modified by the word “about” whether expressly stated or not. “About” as used herein means plus or minus 5% of a numerical value, or more preferably plus or minus 2%. For ranges, the minimum and maximum values are included in the range as well as the values therebetween. It is further understood that the endpoints of each range are significant both in relation to the other endpoint, and independently of the other endpoint. Percentages for concentrations are typically % by weight/weight unless expressly stated otherwise. “Substantially free” means 1% wt/wt or 0.5% wt/wt, or more preferably 0.01% wt/wt or less are present of the identified substance.

The term “ambient conditions” (also known as “room temperature”) as used herein refers to surrounding conditions under about one atmosphere of pressure, at about 50% relative humidity, and at about 25° C., unless otherwise specified. All values, amounts, and measurements described herein are obtained under ambient conditions unless otherwise specified.

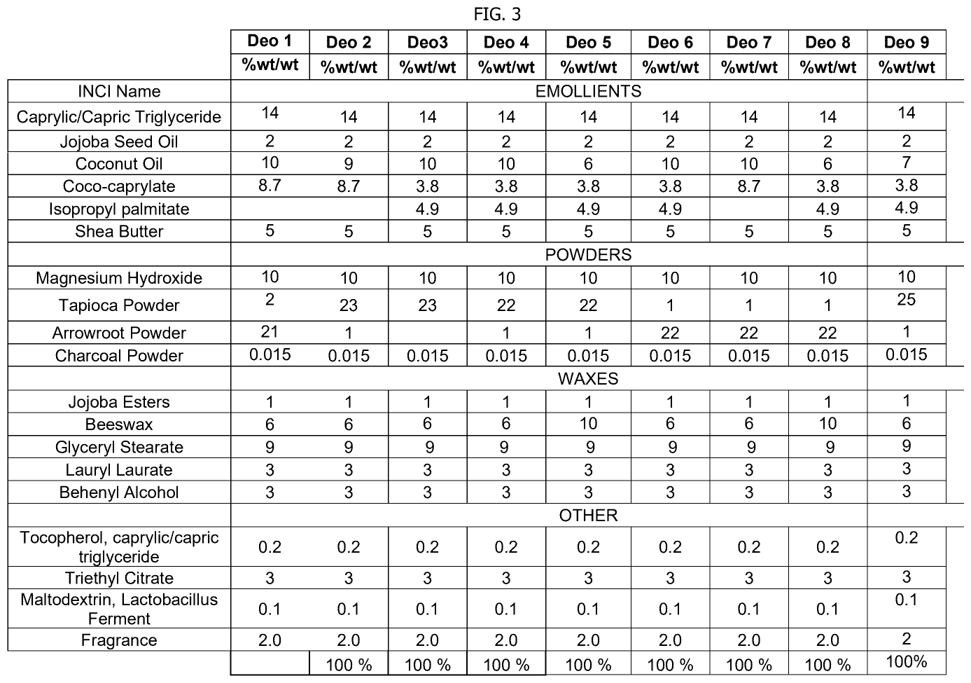

The deodorant compositions disclosed herein are in the form of stick deodorants or paste deodorants that are topically applied to skin and that contain at least 95% by weight of natural or natural-derived ingredients, including but not limited to beeswax, coco-caprylate, shea butter, and coconut oil. Several inventive deodorant compositions are set forth in FIG. 3 as well as in the Example Section below. The deodorants include by percent weight thereof about 1% wt/wt to about 12% wt/wt beeswax, about 10% wt/wt or less coconut oil, about 1% wt/wt to about 10% wt/wt of an esterified oil, at least about 20% wt/wt of a starch, a deodorant agent, and a second wax selected from the group consisting of a jojoba ester, glyceryl stearate, lauryl laurate, behenyl alcohol, and combinations thereof. The coconut oil to beeswax has a ratio in a range of 1:1.5 to 1:3.5, preferably 1:1.67 to 1:3.25. In some embodiments, the beeswax to coconut oil ratio is about 1:2, is about 1:1.67, or is about 1:3.25. In other embodiments, caprylic/capric triglyceride is present and the deodorant compositions have a ratio of coconut oil to beeswax to caprylic/capric triglyceride in a range of 1:1.5:2 to 1:3.5:4.6, more preferably 1:1.67:2 to 1:3.25:4.55. In some embodiments, the coconut oil to beeswax to caprylic/capric triglyceride ratio is about 1:1.67:2, is about 1:2:3.4, or is about 1:3.25:4.55. These ratios are based on the percent weight of each substance in the final deodorant formulations. The deodorant composition is substantially free of water and is free of silicones. In one embodiment, the deodorant composition is in the form of a stick deodorant. The stick deodorant has suitable or desired properties, such as a melting point of 48° C.+/−5° C., a consumer acceptable hardness, effective glide (not sticky), and shelf stability. In another embodiment, the deodorant composition is a paste.

### Emollients

In all embodiments, the composition includes a plurality of emollients. The total emollient concentration is about 30% to about 50%, and more preferably about 30% to about 45% wt/wt of the deodorant composition. In some embodiments, the total emollient concentration is about 30% to 35% wt/wt of the deodorant composition. In other embodiments, the total emollient concentration is about 33% to about 44% wt/wt of the deodorant composition. Natural emollients may be plant or animal based butters, oils, and esters. The total emollient concentration can have 1% to 10%, preferably 3% to 7% wt/wt thereof provided by the natural butters, the balance being the natural oil. In all embodiments, the natural butter can be 5% wt/wt of the total emollient concentration.

Botanical butters are often made from seeds, pips, or nuts of the plant. Some nonlimiting examples of botanical butters include: bacuri butter, cocoa butter, mango butter, and shea butter. Some nonlimiting examples of plant derived oils: acai oil, almond oil, apricot oil, avocado oil, blackberry oil, black cumin seed oil, Brazil nut oil, calendula oil, camellia oil, canola oil, castor oil, coconut oil, corn oil, cranberry oil, cucumber seed oil, hazel nut oil, hemp seed oil, jojoba seed oil, kiwi oil, macadamia nut oil, olive oil, hydrogenated olive oil unsaponifiables, palm oil, palm kernel oil, papaya oil, passion fruit oil, primrose oil, rapeseed oil, rice bran oil, rosehip oil, safflower oil, sunflower seed oil, watermelon seed oil, and wheat germ oil. Additionally, the natural emollient can be a subcomponent of the botanical butter or plant derived oils. One example is fractionated coconut oil, which is essentially comprised of caprylic/capric triglycerides and is in the liquid phase at ambient conditions. Another example is ethyl macadamiate, which is the ester of ethyl alcohol mixed with fatty acids from macadamia nut oil to create an emollient. Other examples include, but are not limited to, hydrogenated ethylhexyl olivate and hydrogenated olive oil unsaponifiables, a plant oil natural emollient available from Clariant under the brand name Plantasens Olive LD. In another embodiment, the emollient can include squalane.

Natural esters include, but are not limited to, coco-caprylate, isopropyl palmitate, and jojoba esters. Jojoba esters are discussed further in the wax section below.

The emollients can include fractionated coconut oil, coconut oil, and coco-caprylate. Fractionated coconut oil is also known as caprylic/capric triglyceride. The concentration of caprylic/capric triglyceride in the deodorant compositions can be about 10% wt/wt to about 18% wt/wt thereof, more preferably about 10% wt/wt to about 16% wt/wt, and even more preferably about 11% to about 15% wt/wt thereof, or even about 12% wt/wt to about 14% wt/wt. In some embodiments, the concentration of caprylic/capric triglyceride is 15% wt/wt or 14% wt/wt, and in others it is about 12% wt/wt. One advantage of utilizing a concentration thereof within any one of these ranges is that it avoids a greasy feel on the skin. Moreover, caprylic/capric triglyceride's distribution of C8 and C10 triglycerides can vary by vendor, such as a 60/40 or 70/30 combination thereof. This variation can affect product consistency and quality.

The concentration of coconut oil in the deodorant composition can be about 0.10% to about 25% wt/wt thereof, more preferably 10% wt/wt or less. Alternately, the concentration of coconut oil can be in a range of 2% wt/wt to 10% wt/wt thereof, and even more preferably about 2% wt/wt to about 8% wt/wt, or even about 3% wt/wt to about 6% wt/wt thereof.

Coco-caprylate is an esterified oil made from coconut oil or palm oil and glycerin or a fatty alcohol or a polyalcohol, but more typically coconut oil and glycerin. Coco-caprylate is a natural ingredient, emollient, and “light” oil that has a non-greasy, non-shiny finish on the skin. By utilizing caprylic/capric triglyceride within any of the stated ranges and introducing ester emollients, such an esterified oil, the resulting stick deodorant tends to have good spreadability and faster skin absorption. Other ester emollients include, but are not limited to, isopropyl palmitate, isononyl isononanoate, ethyl hexyl palmitate, coco-caprylate/caprate, ethyl linoleate, and combinations thereof. Additionally, it was found that the deodorant compositions had improved glide, provided a unique sensorial experience, and good skin absorption. Moreover, the ester emollients have a better lot to lot consistency, which can mitigate the variation contributed by the caprylic/capric triglyceride.

Other suitable emollients include an alkane having a carbon chain length of C₁₃ to C₁₉, coconut alkanes, and combinations thereof, and/or polyglyceryl fatty acid esters. Some example polyglyceryl fatty acid esters include polyglyceryl-x caprylate/caprate, wherein x can be any number between and including 1 to 10. In one embodiment, x equals 10. The fatty acid portion of the polyglyceryl fatty acid esters can have a carbon chain length of 4 to 18. These emollients can modify sensorial attributes of the formulations, provide emulsification power, aid in rise off, and aid in reducing blooming in the formula of the natural butters.

As observed in formulation testing of stick deodorants, when the concentration of coconut oil was greater than 10% wt/wt the deodorant lacked glide (it had too much drag) and it was stickier than desired (even if it met penetration and melt temperature parameters). Waxes, especially with the high amounts being used in the deodorant composition disclosed herein, also contribute to the stickiness and the drag of the deodorant, collectively referred herein as “glide.” It is undesirable for the deodorant to pull hair in the underarm during application (which glide contributes to). Glide was an issue that needed to be addressed. Experiments with new combinations of waxes were performed to improve glide. In one embodiment, the combination of waxes included lauryl laurate and behenyl alcohol.

The high concentration of waxes and the amount of coconut oil need a suitable ratio therebetween for proper glide. Glide is also affected by the high concentration of starch(es) in the deodorant. An esterified oil, such as coco-caprylate, was added to the emollients to replace some or all the coconut oil (with the desire to improve glide). The coco-caprylate can be present as about 1% wt/wt to about 15% wt/wt, more preferably about 2% wt/wt to about 10% wt/wt, and even more preferably about 2% wt/wt to about 5% wt/wt of the deodorant composition. With the addition of coco-caprylate, the coconut oil can be present as about 0.1% wt/wt to about 10% wt/wt of the deodorant composition, preferably 2% wt/wt to about 8% wt/wt thereof, and even more preferably about 3% wt/wt to about 6% wt/wt thereof.

Coco-caprylate is an expensive ingredient, so it was found that good properties can still be achieved by reducing the amount of coco-caprylate and adding another emollient, such as isopropyl palmitate. The coco-caprylate and isopropyl palmitate can be present in a range of ratios of 1:1 to 1:2, more preferably a range of ratios of 1:1.1 to 1:1.4.

The deodorant compositions include one of the natural butters and other oils, such as an esterified oil, in combination with the caprylic/capric triglyceride and the coconut oil. In all embodiments, the emollients can be a combination of caprylic/capric triglyceride, coconut oil, jojoba seed oil, and shea butter. Jojoba seed oil can be present as 1% wt/wt to 10% wt/wt, more preferably 1% wt/wt to 5% wt/wt of the deodorant composition. In all embodiments, jojoba seed oil can be present as 2% wt/wt of the deodorant composition. The shea butter can be present as 1% wt/wt to 10% wt/wt, more preferably 1% wt/wt to 8% wt/wt, and more preferably 3% wt/wt to 7% wt/wt of the deodorant composition. In all embodiments, the shea butter can be about 5% wt/wt of the deodorant composition.

### Powders

The powder is typically a starch. The starch can be selected from the group consisting of corn starch, wheat starch, rice starch, potato starch, tapioca starch, tapioca root powder, arrowroot starch (often called arrowroot powder), maltodextrin, and combinations thereof. Arrowroot powder may be from any one of the following plants Maranta arundinacea, Zamia integrifolia, Tacca leontopetaloides, Pueraria lobata, and Manihot Esculenta. In all embodiments, the arrowroot powder can be from the Maranta arundinacea plants, and the composition can include a mixture of starches, tapioca starch or tapioca root powder and arrowroot powder. In one embodiment, the tapioca starch (Manihot esculenta) or tapioca powder is present in a greater % wt/wt than the arrowroot powder. In another embodiment, the arrowroot powder is present in a greater % wt/wt than the tapioca starch and/or tapioca powder. In all embodiments, the concentration of starch(es) is greater than 20% wt/wt of the composition, more preferably about 21% wt/wt to about 30% wt/wt, and even more preferably about 22% wt/wt to about 27% wt/wt thereof.

In all embodiments, the compositions include a deodorant agent, which can be odor-eliminating and/or antimicrobial ingredients. Antimicrobials may include, without being limited to, beryllium carbonate, magnesium oxide, zinc oxide, zinc stearate, calcium oxide, sodium carbonate, sodium sesquicarbonate, sodium bicarbonate (baking soda), hexamidine, magnesium carbonate, zinc carbonate, zinc ricinoleate, thymol, magnesium hydroxide, magnesium hydroxide and magnesium carbonate hydroxide (dead sea salt), partially carbonated magnesium hydroxide, magnesium stearate, sodium carbonate, calcium carbonate, magnesium carbonate hydroxide, cinnamon essential oil, cinnamon bark essential oil, cinnamic aldehyde, polyvinyl formate, salycilic acid, niacinamide, piroctone olamine, phenoxyethanol, potassium carbonate, potassium bicarbonate, eugenol, linolenic acid, dimethyl succinate, citral, thymol, triethyl citrate, sepiwhite, an substituted or unsubstituted 2-pyridinol-N-oxide material (piroctone olamine), and combinations thereof. The total amount of deodorant agent in any one of the deodorant compositions may be from about 3% to about 20% by weight of the deodorant composition, preferably about 5% to about 15%, and more preferably about 8% to about 13% by weight of the deodorant composition. In all embodiments, the total amount of deodorant agent can be 10% by weight of the deodorant composition. In one embodiment, the deodorant agent is magnesium hydroxide. In another embodiment, the deodorant agent is sodium bicarbonate. Any of the antimicrobials of the present invention may be used as powders, typically having an average particle size of less than 10 μm, more preferably equal to or less than 5 μm.

Suitable natural odor eliminating agents include methylheptylglycerin, Saccharomyces ferment filtrate, and other bacterial ferments. The composition can include one or both thereof but does not include any aluminum based odor eliminating agents. Methylheptylglycerin prevents malodor through the inhibition of the growth of malodor causing bacteria. Methylheptylglycerin is available from Inolex, Inc. under the mark LEXGARD® MHG Natural MB. Saccharomyces ferment filtrate, a natural ingredient obtained by fermenting and filtering yeasts can soothe, hydrate, and protect skin and hair. This functional deodorizing agent uses enzymes created by the fermentation of natural sugar cane or rice. DEOPLEX® natural deodorant is available from Carrubba, Inc. Lactobacillus/Rice Ferment Filtrate is another possible odor eliminating agents. The compositions are free of aluminum-containing compounds and can include or be free of or substantially free of baking soda.

### Waxes

The deodorant compositions disclosed herein have a combination of ester waxes, other waxes, and emollients that together provide a suitable deodorant. The ester wax can be selected from the group consisting of lauryl laurate, lanolin, jojoba esters, glyceryl stearate, and combinations thereof. In some embodiments, the plurality of waxes is free of or substantially free of stearyl stearate, palmityl stearate, stearyl behenate, cetearyl behenate, and behenyl behenate; but, in other embodiments one or more of these can be present.

Other suitable waxes, especially natural waxes, include but are not limited to sunflower wax, candelilla wax, carnauba wax, bayberry wax, castor wax (also known as hydrogenated castor oil), beeswax, rapeseed wax, rice bran wax, and combinations thereof. In one embodiment, beeswax is present and can be present in a range of 1% wt/wt to 12% wt/wt, more preferably 2% wt/wt to 110% wt/wt, and even more preferably 5% wt/wt to 10% wt/wt. Other natural waxes are optionally present and can be present typically in a range of 0% wt/wt to 5% wt/wt.

The wax portion of the deodorant composition can be a plurality of waxes. The plurality of waxes (totaled together) can be present as about 15% wt/wt to about 30% wt/wt, preferably about 18% wt/wt to about 28% wt/wt, and even more preferably about 21% wt/wt to about 27% wt/wt of the deodorant composition. In all embodiments, the plurality of waxes (totaled together) can be present as about 22% wt/wt of the deodorant composition. In all embodiments, the plurality of waxes (totaled together) can be present as about 26% wt/wt of the deodorant composition. The plurality of waxes contributes a high amount of wax to the composition. As discussed above, in such quantities, the wax portion can affect the “glide” property of the stick deodorant. As discussed above, it was found that the addition of the emollient coco-caprylate (such as CETIOL® C 5 available from BASF) improved the glide property.

In a first embodiment, the wax portion includes beeswax (Cera Alba). In a second embodiment, the wax portion of the deodorant composition is a combination of waxes that includes the ester wax, glyceryl stearate. In a third embodiment, the wax portion includes beeswax and glyceryl stearate. In a fourth embodiment, the wax portion includes beeswax, jojoba esters, and glyceryl stearate. In a fifth embodiment, the wax portion includes beeswax, jojoba esters, and lauryl laurate. In a six embodiment, the wax portion includes glyceryl stearate and jojoba esters. In a seventh embodiment, the wax portion includes glyceryl stearate and lauryl laurate. In an eight embodiment, the wax portion includes glyceryl stearate and behenyl alcohol. In a ninth embodiment, the wax portion includes glyceryl stearate, lauryl laurate, and jojoba esters. In a tenth embodiment, the wax portion includes glyceryl stearate, behenyl alcohol, and jojoba esters. In an eleventh embodiment, the wax portion includes beeswax, glyceryl stearate, and lauryl laurate. In all embodiments, the combination can include beeswax, jojoba esters, glyceryl stearate, and lauryl laurate.

The jojoba esters can be present as 0.1% to about 10% wt/wt, more preferably 0.1% wt/wt to about 5% wt/wt of the deodorant composition. In all embodiments, the jojoba esters can be present as 1% wt/wt of the deodorant composition. The beeswax can be present as about 1% wt/wt to about 15% wt/wt, more preferably 3% wt/wt to about 10% wt/wt of the deodorant composition. In all embodiments, the beeswax can be present as 6% wt/wt of the deodorant composition or as 10% wt/wt of the deodorant composition. The glyceryl stearate can be present as about 1% wt/wt to about 15% wt/wt, more preferably 3% wt/wt to about 13% wt/wt of the deodorant composition. In all embodiments, the glyceryl stearate can be present as 9% wt/wt of the deodorant composition or as 12% wt/wt of the deodorant composition.

Among the other waxes, a fatty alcohol wax/emollient can be selected from the group consisting of stearyl alcohol, behenyl alcohol, cetyl alcohol, oleyl alcohol, arachidyl alcohol, lauryl alcohol, myristyl alcohol, 1-decanol, 1-octanol, a fatty alcohol having a carbon chain length of up to and including 14 carbons, and combination thereof. In some embodiments, the carbon chain length is 4 to 20 carbons. In some embodiments, the carbon chain length is 20 or more. A fatty alcohol that is a liquid at room temperature enables mixing without a heating step, which may be desired for ease of manufacturing. In some embodiments, stearyl alcohol is not present, i.e., the deodorant compositions are free of or substantially free of stearyl alcohol. In all embodiments, the fatty alcohol wax/emollient can be present as about 3% wt/wt or more of the deodorant composition, but typically not more than about 15% wt/wt, more preferably not more than 10% wt/wt, and even more preferably not more than 5% wt/wt of the deodorant composition.

### Other

The compositions can include other ingredients selected from the group consisting of absorbents, antioxidants, antiperspirant agents, anti-whitening agents, coloring agents, essential oils, fragrances, pH adjusters, preservatives, vitamins etc. Other ingredients are typically present as about 10% wt/wt or less of the composition. In one embodiment, the other ingredients, collectively, are about 6% wt/wt or less of the composition. In one embodiment, the other ingredients, collectively, are about 5% wt/wt of the composition. In FIG. 3, the substances listed as “other ingredients” include, but are not limited to, tocopherol, triethyl citrate, fragrances, and maltodextrin, Lactobacillus Ferment. Charcoal can also be included in the category of “other ingredient” since it is an absorbent/coloring agent and is present as less than 0.10% wt/wt of the composition.

In embodiments that include an antiperspirant or antiperspirant-like agent, it is desirable to include one that is aluminum free and natural or naturally derived, such as in accordance with ISO 16128. The antiperspirant or antiperspirant-like agent may be or include Chinese sumac galls, turmeric, schisandra, ephedra root, mulberry leaf, Amur cork tree bark, rough cocklebur (Xanthium strumarium) liniment, common yarrow, soapwort, elderberry flowers, sage, horsetail (Equisetum arvense), rockrose (Cistus ladanfer), Milkvetch (Astragalus spp.), comfrey, Himalayan cedar, nut grass, jasmine, black turmeric, vetiver, and combinations thereof. Any of these antiperspirant agents can be applied topically to reduce sweat or control sweating. In some embodiments, the antiperspirant or antiperspirant-like agent is an extract from one or a combination of these antiperspirant or antiperspirant-like agents or other plants that can provide an antiperspirant effect. While the preferred antiperspirant or antiperspirant-like agent includes natural or naturally derived antiperspirant agents, the antiperspirant or antiperspirant-like agent can be a synthetic agent.

Fragrances are optional because some consumers like fragrance free deodorants. The fragrance can be present in an amount of 0.1% wt/wt to 5%, preferably 0.3% wt/wt to 3% by wt/wt, and even more preferably about 2% wt/wt of the deodorant composition. Any fragrance used herein is at least 50% natural, preferably at least 75% natural, at least 80% natural, at least 90% natural, and even more preferably at least 95% natural.

In all aspects, the compositions are either free of silicone(s) or substantially free of silicone(s). In all aspects, the compositions are free of or substantially free of water. In all aspects, the compositions as manufactured are either free or substantially free of aluminum or aluminum containing substances, petrolatum, petroleum jelly, parabens, phthalates, sulfates, DMDM Hydantoin (1,2-Dimethylol-5,6-dimethylhydantoine), FD&C Colors (Food Drug & Cosmetic), Mineral Oil, Methylisothiazolinone/Methylchloroisothiazolinone (MIT/CIT), nitro- and polycyclic musks, Polyethylene Glycols (PEGs), soybean oil, siloxane based ingredients, stearyl alcohol, Opuntia ficus-indica seed oil, sunflower seed oil, and sunflower seed wax.

### EXAMPLES

Suitable deodorant formulations 1-9 are provided in FIG. 3. Each deodorant composition was evaluated for suitable stick deodorant properties, such as needle penetration with Og weight, melting point, glide versus stickiness, short term stability, and long-term stability.

Additional deodorant formulations 10-12 are presented in Table 1 below.

TABLE 1
Suitable Deodorant Formulations

| | Deo 10 | Deo 11 | Deo 12 |
|---|---|---|---|
| | % wt/wt | % wt/wt | % wt/wt |
| Caprylic/Capric Triglyceride | 12 | 14 | 14 |
| Jojoba Seed Oil | 2 | 2 | 2 |
| Coconut Oil | 6 | 3.1 | 4.1 |
| Coco-caprylate | 3.4 | 3.8 | 3.8 |
| Isopropyl palmitate | 4.5 | 4.9 | 4.9 |
| Shea Butter | 4.6 | 5 | 5 |
| Magnesium Hydroxide | 10 | 10 | 10 |
| Tapioca Powder | 25 | 25 | 25 |
| Arrowroot Powder | 1 | 1 | 1 |
| Charcoal Powder | 0.015 | 0.015 | 0.015 |
| Jojoba Esters | 0.6 | 1 | 2 |
| Beeswax | 10 | 10 | 8 |
| Glyceryl Stearate | 8 | 9 | 9 |
| Lauryl Laurate | 2.6 | 3 | 3 |
| Behenyl Alcohol | 4 | 3 | 3 |
| Tocopherol, caprylic/capric triglyceride | 0.2 | 0.2 | 0.2 |
| Triethyl Citrate | 4 | 3 | 3 |
| Lactobacillus Ferment | 0.02 | 0.2 | 0.02 |
| Fragrance | 2.0 | 2 | 2.0 |
| | 100% | 100% | 100% |

In another embodiment, the deodorant formulation includes stearyl alcohol. Table 2 below includes a column with ranges for the ingredients as well as Deodorant Formulations 13 and 14.

TABLE 2

| | % wt/wt | Deo 13 | Deo 14 |
|---|---|---|---|
| | ranges | % wt/wt | % wt/wt |
| First emollient | 12-18 | 15 | 15 |
| (Caprylic/Capric Triglyceride) | | | |
| Second emollient | 12-24 | 16 | 17 |
| Jojoba Seed Oil | 1-5 | 2 | 2 |
| Coconut Oil | 3-10 | 4 | 5 |
| Shea Butter | 3-10 | 5 | 3.7 |
| Deodorant agent | 5-15 | 10 | 10 |
| Tapioca Powder | 0-27 | 23.7 | 22 |
| Arrowroot Powder | 0-27 | 0 | 1 |
| Charcoal Powder | 0-0.5 | 0.015 | 0.015 |
| Jojoba Esters | 0-5 | 1 | 1 |
| Beeswax | 2-12 | 6 | 6 |
| Stearyl Alcohol | 0-20 | 12 | 12 |
| Tocopherol, | 0-1 | 0.2 | 0.2 |
| caprylic/capric triglyceride | | | |
| Triethyl Citrate | 0-6 | 3 | 3 |
| Lactobacillus Ferment | 0-1 | 0.1 | 0.1 |
| Fragrance | 0-6 | 2 | 2 |
| | | 100% | 100% |

The secondary emollient is selected from the group consisting of an oil, a liquid fatty alcohol, a fatty acid, squalane, and a combination thereof. The oil can be a vegetable or plant derived oil. In some embodiments the oil is selected from the group consisting of safflower oil, corn oil, sweet almond oil, argan oil, grapeseed oil, olive oil, avocado oil, palm oil, palm kernel oil, chia seed oil, rice bran oil, and combinations thereof. The fatty acid is selected from the group consisting of linolenic acid, linoleic acid, oleic acid, myristic acid, palmitic acid, stearic acid, capric acid, caprylic acid, lauric acid, behenic acid, and combinations thereof. The liquid fatty alcohol can be one or more of those discussed above with respect to all deodorant formulations.

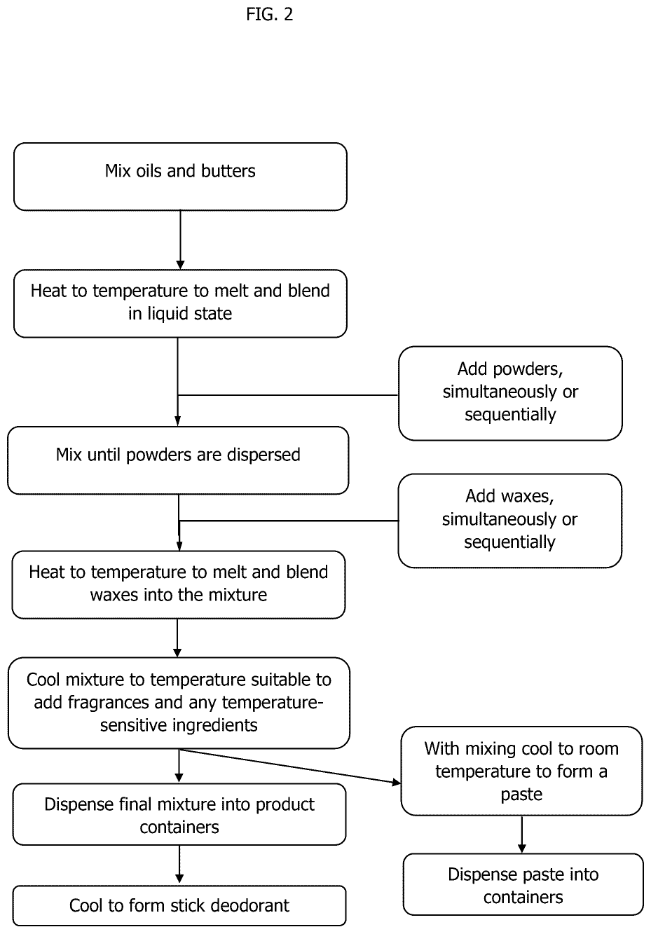

Trial 4 from FIG. 3 is reproduced below (with the fragrance omitted) with Phases A-D recited therein for ease of reference relative to the method of making the deodorant compositions according to the method of FIG. 2.

TABLE 3

| | Deo 4 | | Deo 4 |
|---|---|---|---|
| | % wt/wt | | % wt/wt |
| Phase A | | Phase B | |
| Caprylic/capric | 14 | Deodorant agent | 10 |
| triglyceride | | | |
| Jojoba seed oil | 2 | Tapioca starch | 22 |
| Coconut oil | 10 | Arrowroot Powder | 1 |
| Coco-caprylate | 3.8 | Charcoal powder | 0.015 |
| Isopropyl Palmitate | 4.9 | | |
| Shea butter | 5 | | |
| Phase C | | Phase D | |
| Glyceryl stearate | 9 | Tocopherol | 0.2 |
| Jojoba esters | 1 | Triethyl citrate | 3 |
| Beeswax | 6 | Maltodextrin, | 0.1 |
| | | lactobacillus ferment | |
| Lauryl laurate | 3 | | |
| Behenyl alcohol | 3 | | |

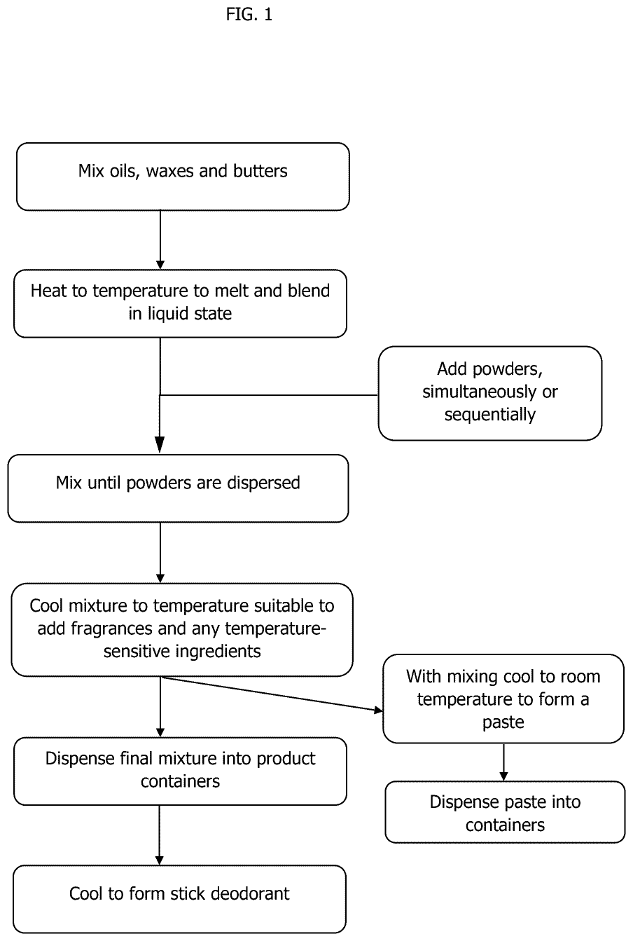

With reference to FIG. 1, the compositions disclosed herein are made by mixing with heat the oils, butters, and waxes. The temperature for heating the waxes may be in a range of about 75° C. to about 90° C., preferably about 80° C. to about 85° C. The powders can be added to the heated mixture with stirring sequentially or simultaneously. If the addition is simultaneous, the powders can be mixed before addition to the heated mixture. The powders are mixed therein until evenly dispersed throughout (the mixture should be smooth and generally without grains of powder). In one exemplary method, a high flow or high shear mixer can be used, and optionally, a screen or screens can be utilized, which can help break down powder clumps. The heated mixture with the dispersed powders is cooled, aided by the addition of the powders themselves, to allow the addition of fragrance and temperature-sensitive materials. The temperature for the addition of the temperature-sensitive materials may be in a range of about 55° C. to about 70° C., preferably about 60° C., or any temperature suitable for a specific temperature sensitive material. Next, the final mixture can be cooled further before and/or after being dispensed into product containers.

In one exemplary method, the filled product containers can be placed into a cooling chamber. Cooling is conducted at a temperature and time duration suitable for the structure of the stick deodorant to set. Typically, the cooling is deemed complete when a center core temperature of the stick deodorant is equal to or less than 40° C.

With reference to FIG. 2, the compositions disclosed herein are made by mixing with heat the oils and butters. The temperature for heating may be about 85° C. The powders can be added to the heated mixture with stirring sequentially or simultaneously. If the addition is simultaneous, the powders can be mixed before addition to the heated mixture. The powders are mixed therein until evenly dispersed throughout (the mixture should be smooth and generally without grains of powder). In one exemplary method, a high-flow or high-shear mixer can be used, and optionally, a screen or screens can be utilized, which can help break down powder clumps. Next, the waxes, simultaneously or sequentially, are added with mixing to the heated mixture with the dispersed powders until the waxes are melted and mixed therein. The mixing can occur in the same high-flow or high-shear mixer. The resulting mixture is cooled to allow the addition of fragrance and temperature-sensitive materials. The temperature may be in a range of about 60° C. to about 70° C., or any temperature suitable for a specific temperature-sensitive material. Next, the final mixture can be cooled further before and/or after being dispensed into product containers.

In one exemplary method, the filled product containers can be placed into a cooling chamber. Cooling is conducted at a temperature and time duration suitable for the structure of the stick deodorant to set. Typically, the cooling is deemed complete when a center core temperature of the stick deodorant is equal to or less than 40° C.

In either variation of the method, the starch powders can be added without further heat, which can serve to quench the mixture.

### Paste Deodorants

In another aspect, rather than forming a stick deodorant, a paste deodorant can be made from any of the above compositions. The preferred method of making a paste deodorant includes heating oil and butter ingredients, and optionally a wax, to form a heated mixture, mixing a starch powder into the heated mixture, cooling to a preselected temperature for addition of a temperature sensitive ingredient, adding the temperature sensitive ingredient, and cooling to room temperature with mixing to form a paste deodorant. The paste deodorant can then be dispensed into containers. The container may be a jar or a squeeze tube.

The paste deodorant can be anhydrous, just like the stick deodorants described herein, and may be described as a very thick oil emulsion.

The heating of the wax separately may occur separately from the oils and butters. When the wax is heated separately, the wax can be added after the starch powder with mixing.

The addition of the starch powder may include addition of a plurality of starch powders, which can occur simultaneously or sequentially.

The deodorant formulations disclosed herein provide the advantage of being a product that is 95% or more “natural” as discussed herein, which is desirable for many consumers. Moreover, the resulting deodorant, whether stick or paste, has consumer acceptable properties, such as, but not limited to glide, fragrance, a smooth body (e.g., an absence of cracks when in the form of a stick) that is suitable for advancement within the container during use, etc.

It should be noted that the embodiments are not limited in their application or use to the details of construction and arrangement of parts and steps illustrated in the drawings and description. Features of the illustrative embodiments, constructions, and variants may be implemented or incorporated in other embodiments, constructions, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention. Having described the invention in detail and by reference to preferred embodiments thereof, it will be apparent that modifications and variations are possible without departing from the scope of the invention which is defined in the appended claims.

What is claimed is:

1. A deodorant composition comprising:
about 1% wt/wt beeswax to about 12% wt/wt beeswax;
about 10% wt/wt or less coconut oil;
about 1% wt/wt to about 10% wt/wt of an esterified oil;
at least 20% wt/wt of a starch;
a deodorant agent; and
a second wax selected from the group consisting of a glyceryl stearate, lauryl laurate, behenyl alcohol, and combinations thereof;
wherein the deodorant composition comprises at least 95% by weight natural or natural-derived ingredients, and is in the form of a stick deodorant.

2. The deodorant composition of claim 1, wherein the esterified oil comprises coco-caprylate.

3. The deodorant composition of claim 1, wherein the deodorant composition is substantially free of water and is free of silicones.

4. The deodorant composition of claim 1, further comprising about 18% wt/wt or less caprylic/capric triglycerides.

5. The deodorant composition of claim 4, wherein the concentration of caprylic/capric triglyceride is about 10% wt/wt to about 16% wt/wt of the deodorant composition.

6. The deodorant composition of claim 1, wherein the second wax is a combination of glyceryl stearate, lauryl laurate, and behenyl alcohol.

7. The deodorant composition of claim 6, wherein the glyceryl stearate, lauryl laurate, and behenyl alcohol are present in a ratio of 3:1:1 to about 3:1.5:1.

8. The deodorant composition of claim 2, further comprising isopropyl palmitate.

9. The deodorant composition of claim 8, wherein the coco-caprylate and isopropyl palmitate are present in a range of ratios of 1:1 to 1:2.

10. The deodorant composition of claim 9, wherein the coco-caprylate and isopropyl palmitate are present in a range of ratios of 1:1.1 to 1:1.4.

11. The deodorant composition of claim 1, wherein the starch comprises tapioca starch and/or arrowroot powder.

12. The deodorant composition of claim 1, wherein the starch comprises a mixture of tapioca starch and arrowroot powder.

13. The deodorant composition of claim 1, further comprising shea butter, which is present in a range of 1% wt/wt to 8% wt/wt of the deodorant composition.

14. The deodorant composition of claim 1, wherein the second wax comprises glyceryl stearate and/or lauryl laurate.

15. The deodorant composition of claim 14, wherein the second wax comprises both glyceryl stearate and lauryl laurate.

16. The deodorant composition of claim 15, wherein the second wax further comprises the jojoba ester.

17. The deodorant composition of claim 1, further comprising a fatty alcohol selected from the group consisting of behenyl alcohol, cetyl alcohol, oleyl alcohol, arachidyl alcohol, ceryl alcohol, and combination thereof.

18. The deodorant composition of claim 1, wherein the deodorant agent is selected from the group consisting of beryllium carbonate, magnesium carbonate, calcium carbonate, magnesium hydroxide, magnesium hydroxide and magnesium carbonate hydroxide, magnesium stearate, partially carbonated magnesium hydroxide, piroctone olamine, potassium carbonate, potassium bicarbonate, sodium carbonate, sodium sesquicarbonate, sodium bicarbonate, hexamidine, zinc carbonate, thymol, polyvinyl formate, salycilic acid, niacinamide, magnesium carbonate hydroxide, cinnamon essential oil, cinnamon bark essential oil, cinnamic aldehyde, magnesium oxide, zinc oxide, zinc stearate, and combinations thereof.

19. The deodorant composition of claim 1, further comprising triethyl citrate and/or a fragrance.

20. The deodorant composition of claim 19, wherein the fragrance is present and comprises about 0.1% wt/wt to about 5% wt/wt of the deodorant composition.

21. The deodorant composition of claim 1, further comprising about 1% wt/wt or less than 1% wt/wt each of other ingredients selected from the group consisting of tocopherol, maltodextrin Lactobacillus ferment, a lubricating powder, and combinations thereof.
